(19) United States
(12) Patent Application Publication
(10) Pub. No.: US 2006/0253057 A1
(43) Pub. Date: Nov. 9, 2006

(54) Buckling restrained structural brace assembly

(76) Inventors: Xiaoxuan Qi, Moraga, CA (US); Jiehua Jay Shen, Ames, IA (US)

Correspondence Address: XIAOXUAN QI, 351 DONALD DRIVE, MORAGA, CA, 94556, US

(21) Appl. No.: 11/408,343

(22) Filed: Apr. 22, 2006

Related U.S. Application Data
(60) Provisional application No. 60/677,854, filed on May 4, 2005.

(51) Int. Cl. A61F 5/00

(52) U.S. Cl. 602/23

(57) ABSTRACT

A buckling restrained structural brace assembly including a load-bearing member and a buckling-restraining member to prevent the load-bearing member from buckling in extreme loading conditions. Fins can be attached to either the load-bearing member, where the fins are spaced apart from the buckling-restraining member by less than one inch, or the fins can be attached to the surrounding buckling-restraining member where the fins are spaced apart from the load-bearing member by less than one inch. Alternately, there may be no fins attached to the load-bearing member or to the buckling-restraining member. In such case, the load-bearing member is substantially surrounded by a buckling-restraining member to prevent the load-bearing member from buckling. The buckling-restraining member may be spaced apart from the load-bearing member by preferably a distance of less than one inch.

This application claims the priority to Provisional Application No. 60/677854 filed on May 4, 2005

### BACKGROUND OF THE INVENTION

The present invention relates generally to structural brace assemblies. More specifically, the present invention relates to a buckling restraining member surrounding a structural brace member subjected to axial tension and compression.

The knowledge and techniques used in earthquake engineering have advanced rapidly in the last few decades. As a result, the number of casualties suffered during earthquakes has been greatly reduced. Nevertheless, observations of recent major earthquakes indicate that under performed structural elements in buildings and other structures result in a significant amount of structural and nonstructural damage, causing billions of dollars of loss in property damages and business interruptions.

Structures are susceptible to large lateral displacements during severe earthquake ground motions. These displacements can cause damage to a structure in a variety of ways. The displacements can damage the beam to column connections within a structure, making the structure unstable. The displacements can also buckle the bracing members, causing them to lose the load bearing capacities required to support the structure.

Braces provide stiffness and strength to a structure. Effective braces should exhibit nearly equal stiffness and strength in tension and compression, and be able to undergo many tension-compression inelastic deformation cycles without losing their load carrying capacity. Conventional brace members usually have less compression strength than tension strength because the members have a tendency to buckle under compression. Once a brace has buckled, its stiffness and strength are reduced greatly. Also, a buckled brace may rupture prematurely during inelastic deformation cycles. By restraining the buckling of a brace member, the compressive load that the member can carry is increased, and its energy dissipation capacity is enhanced.

Conventional attempts to increase the compressive strength of bracing members encapsulate a load-bearing member in concrete or mortar material and a sleeve. While such members can resist compressive loads without buckling, they have a number of disadvantages. They are expensive to manufacture because of the involvement of different types of materials and the need to position the concrete or mortar material precisely, and are generally manufactured at a facility remote to the installation. They are heavy because of the mass of concrete or mortar they contain. Because the load bearing members are encapsulated in solid materials, they are very difficult to inspect after a significant loading event. If such a structural member becomes damaged, it may be difficult to replace because of the weight and size of the member.

### SUMMARY OF THE INVENTION

The present invention is directed to a buckling restrained structural brace assembly that provides for a simple and economical way to enhance the seismic performance of braces.

A buckling restrained structural brace assembly consists of a load-bearing member and a buckling-restraining member that prevents the load-bearing member from buckling. The buckling-restraining member preferably substantially surrounds the load-bearing member. One, or more than one fin can be attached to the load-bearing member, the fin or fins being preferably spaced apart from the buckling-restraining member by a distance of less than one inch.

The load-bearing member may be composed of a material selected from the group including steel and aluminum. Likewise, the buckling-restraining member may be composed of a material selected from the group including steel and aluminum.

The buckling-restraining member may contain one, or more than one opening for inspection of the load-bearing member. The buckling-restraining member may be one element substantially surrounding the load-bearing member, or may be made up of more than one element affixed together to form a rigid member substantially surrounding the load-bearing member.

As opposed to one, or more than one fin being attached to the load-bearing member, the fin or fins may be attached to the buckling-restraining member, the fin or fins being spaced apart from the load-bearing member preferably by a distance of less than one inch.

Alternately, there may be no fins attached to the load-bearing member or to the buckling-restraining member. In such case, the load-bearing member is substantially surrounded by a buckling-restraining member to prevent the load-bearing member from buckling. The buckling-restraining member may be spaced apart from the load-bearing member by preferably a distance of less than one inch.

Additional features and advantages of the present invention are described in, and will be apparent from, the following Detailed Description of the Invention and the figures.

### BRIEF DESCRIPTION OF THE FIGURES

FIG. 1 is a front elevational view of an embodiment of a buckling restrained structural brace assembly of the present invention.

FIG. 2 is a front elevational view of an embodiment of a buckling restrained structural brace assembly of the present invention with inspection ports.

FIG. 3 is a cross-sectional view of an embodiment of the buckling restrained structural brace assembly of FIG. 1.

FIG. 4 is a cross-sectional view of an embodiment of the buckling restrained structural brace assembly of the present invention.

FIG. 5 is a cross-sectional view of an embodiment of the buckling restrained structural brace assembly of the present invention.

FIG. 6 is a cross-sectional view of an embodiment of the buckling restrained structural brace assembly of FIG. 1.

FIG. 7 is a cross-sectional view of an embodiment of the buckling restrained structural brace assembly of the present invention.

FIG. 8 is a cross-sectional view of an embodiment of the buckling restrained structural brace assembly of the present invention.

FIG. 9 is a cross-sectional view of an embodiment of the buckling restrained structural brace assembly of the present invention.

FIG. 10 is a cross-sectional view of an embodiment of the buckling restrained structural brace assembly of the present invention.

FIG. 11 is a cross-sectional view of an embodiment of the buckling restrained structural brace assembly of the present invention.

FIG. 12 is a cross-sectional view of an embodiment of the buckling restrained structural brace assembly of the present invention.

FIG. 13 is a cross-sectional view of an embodiment of the buckling restrained structural brace assembly of the present invention.

FIG. 14 is a cross-sectional view of an embodiment of the buckling restrained structural brace assembly of the present invention.

FIG. 15 is a cross-sectional view of an embodiment of the buckling restrained structural brace assembly of the present invention.

FIG. 16 is a cross-sectional view of an embodiment of the buckling restrained structural brace assembly of the present invention.

FIG. 17 is a partial cross-sectional view of an embodiment of the buckling restrained structural brace assembly of FIG. 1.

FIG. 18 is a partial cross-sectional view of an embodiment of the buckling restrained structural brace assembly of the present invention.

FIG. 19 is a partial cross-sectional view of an embodiment of the buckling restrained structural brace assembly of the present invention.

FIG. 20 is a partial cross-sectional view of an embodiment of the buckling restrained structural brace assembly of the present invention.

FIG. 21 is a partial cross-sectional view of an embodiment of the buckling restrained structural brace assembly of the present invention.

FIG. 22 is a partial cross-sectional view of an embodiment of the buckling restrained structural brace assembly of the present invention.

FIG. 23 is a partial cross-sectional view of an embodiment of the buckling restrained structural brace assembly of the present invention.

FIG. 24 is a partial cross-sectional view of an embodiment of the buckling restrained structural brace assembly of the present invention.

FIG. 25 is a partial cross-sectional view of an embodiment of the buckling restrained structural brace assembly of the present invention.

FIG. 26 is a partial cross-sectional view of an embodiment of the buckling restrained structural brace assembly of the present invention.

### DETAILED DESCRIPTION OF THE INVENTION

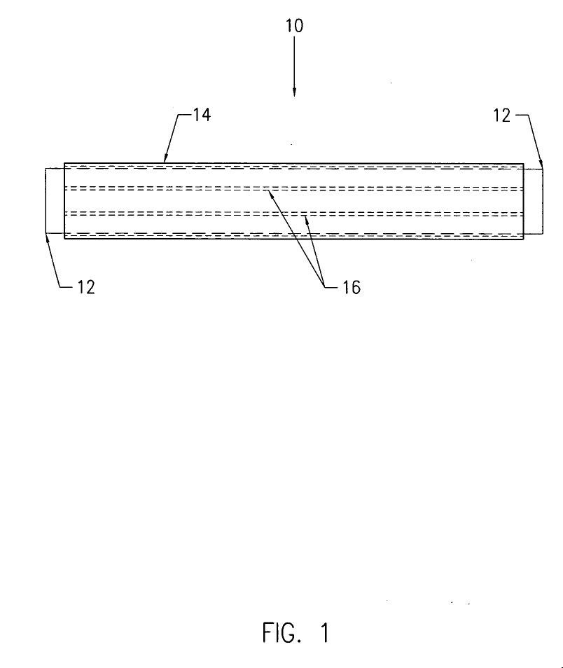

A buckling restrained structural brace assembly (BRSBA 10) is a load-bearing member (core 12) preferably surrounded by a buckling-restraining member (shell 14). An embodiment of the present invention resists applied loads primarily in the form of axial tension and compression. FIG. 1 illustrates an embodiment of the present invention. The buckling restrained structural brace assembly 10 may include a core 12 to which fins 16 are attached by welding or any other suitable method of fastening such as banding, riveting, bolting, or screwing. The fins 16 may also be integrally formed with the core 12. This core 12 and fin 16 element will expand when the BRSBA 10 is subjected to compression. The core 12 and fin 16 element are surrounded by a shell 14. In one embodiment, this shell 14 preferably substantially surrounds the core 12 and fins 16 without touching the fins 16. The shell 14 will come into contact with the fins 16 upon the core 12 being subjected to compressive load, where the shell 14 will prevent the core 12 from buckling. In an embodiment of the buckling restrained structural brace assembly 10, the shell 14 is a single member. The shell 14 may be rolled hollow sections (seamed or seamless), or built up of steel plates.

Preferably, the brace is formed of structural steel or other suitable metal alloys. It should be appreciated that the brace may also be formed of other suitable material including aluminum, titanium, brass, bronze, iron and composite materials formed of materials such as metals, ceramics, glasses, and polymers.

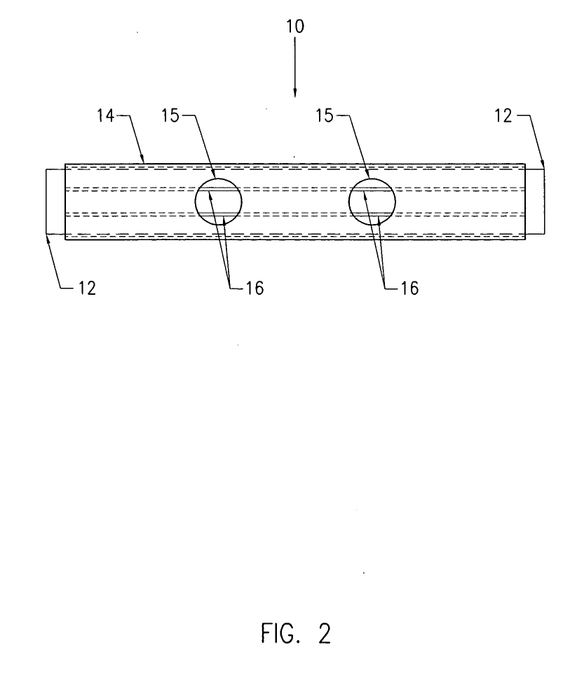

FIG. 2 depicts an embodiment of the present invention including one or more inspection ports 15 through the shell 14. In an embodiment of the BRSBA 10, the inspection port 15 would be used to inspect the core 12 and fins 16 for permanent deformation or damage subsequent to being subjected to a load. Although the inspection ports 15 are depicted as circle in shape, the size, shape, and configuration of the inspection port 15 is limited only by functionality. The BRSBA 10 in an embodiment includes covers (not shown) on the inspection port 15 and connecting ends of the BRSBA 10 to prevent the accumulation of dirt and debris within the shell 14 and to prevent infestation by insects or nest building birds.

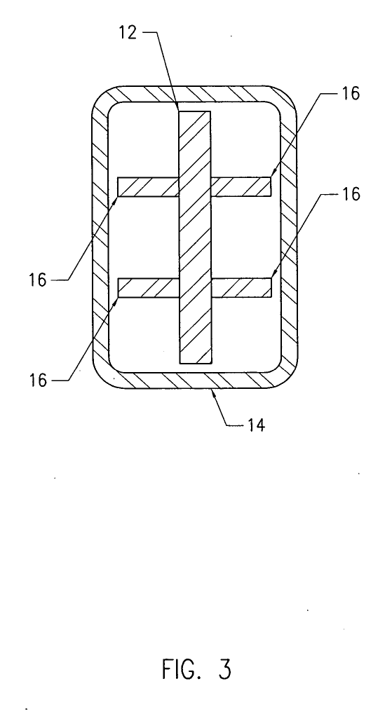

FIG. 3 illustrates a cross-sectional view of an embodiment of the buckling restrained structural brace assembly 10. The fins 16 are attached to the core 12. Although the cross-section of the core 12 is rectangular in FIG. 3, the size, shape, and configuration of the core 12 is limited only by functionality. The fins 16 may also be integrally formed with the core 12. The fins 16 are preferably sufficiently long to come into close proximity to the shell 14, but not long enough to contact the shell 14. Although the shell 14 is shown in FIG. 1 as rectangular, the size, shape, and configuration of the shell 14 is limited only by functionality. Additionally, the shell 14 preferably does not come into contact with the core 12. In this embodiment of the present invention, the shell 14 is a single member.

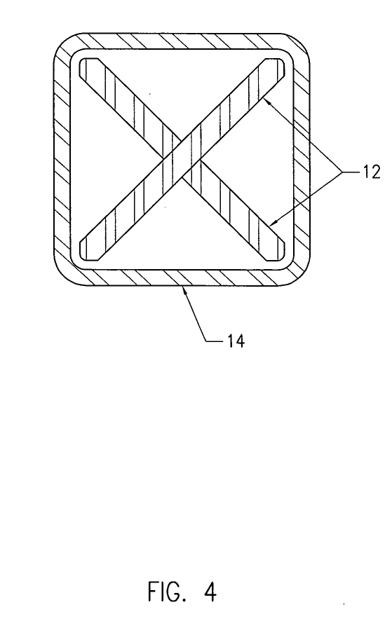

FIG. 4 is a cross-sectional view which illustrates another embodiment of the BRSBA 10. Here, the core 12 is preferably a cruciform shape and the fins (not shown) are absent. When subjected to loading, the core 12 comes into direct contact with the shell 14 which prevents the core 12 from buckling.

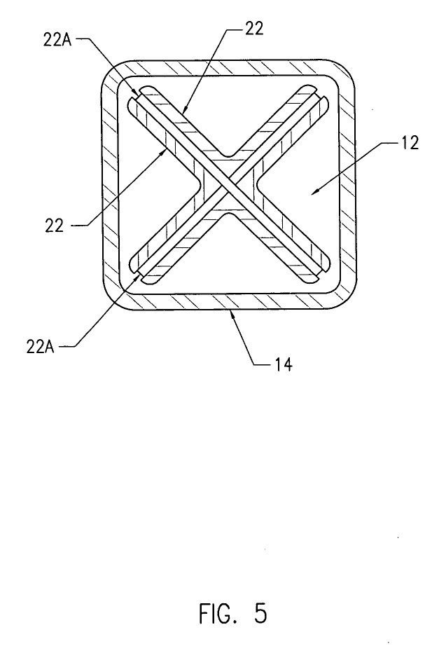

FIG. 5 depicts a cross-sectional view of another embodiment of the present invention. Here, the cruciform shaped core 12 is preferably comprised of four lengths of angle 22 oriented longitudinally, with the bend of the angle 22 directed toward the longitudinal axis of the core 12. The lengths of angle 22 are separated from each with spacer plates 22.

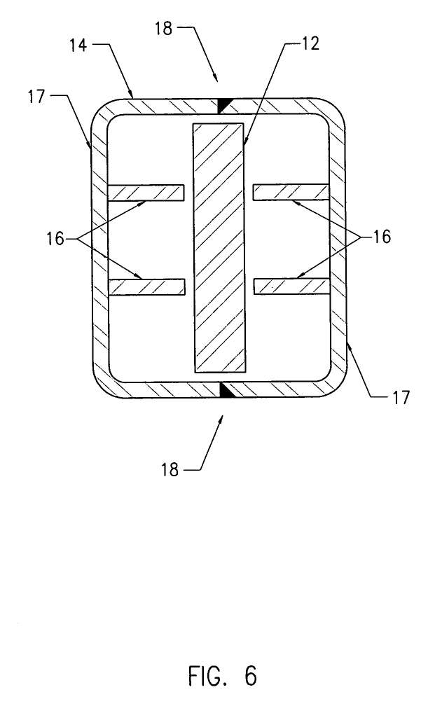

FIG. 6 depicts a cross-sectional view of another embodiment of the present invention. Here, the shell 14 includes two shell halves 17, connected by a shell connection 18. The fins 16 are attached to the interior surface of the shell halves 17 by welding or any other suitable method of fastening. The fins 16 may also be integrally formed with the shell halves 17. Preferably, the fins 16 attached to the shell halves 17 do not come into contact with the core 12, but preferably come into close proximity to the core 12.

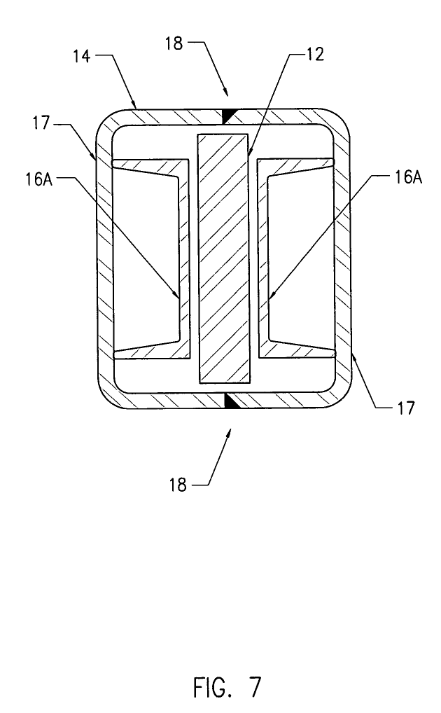

FIG. 7 is a cross-sectional view of another embodiment of the present invention. Here, the shell 14 includes two shell halves 17, connected by a shell connection 18. Fins in an alternate form of channels 16A are attached to the interior surface of the shell halves 17 by welding or any other suitable method of fastening. Preferably, the channels 16A attached to the shell halves 17 do not come into contact with the core 12, but preferably come into close proximity to the core 12.

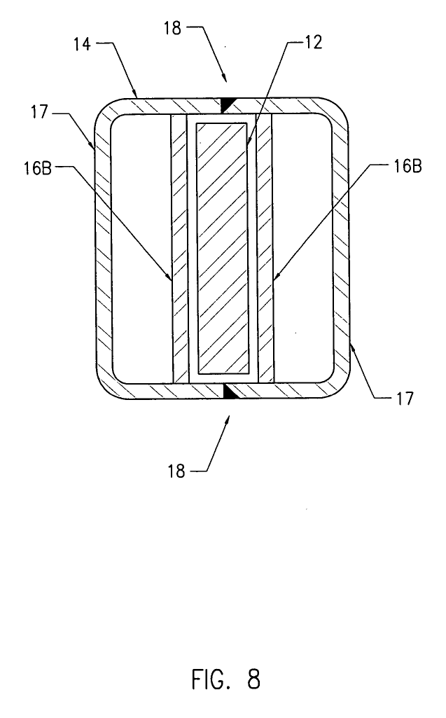

FIG. 8 is a cross-sectional view of another embodiment of the present invention. Here, the shell 14 includes two shell halves 17, connected by a shell connection 18. Diaphragm plates 16B, rather than fins, are attached to the interior surface of the shell halves 17 by welding or any other suitable method of fastening. Preferably, the diaphragm plates 16B attached to the shell halves 17 do not come into contact with the core 12, but preferably come into close proximity to the core 12.

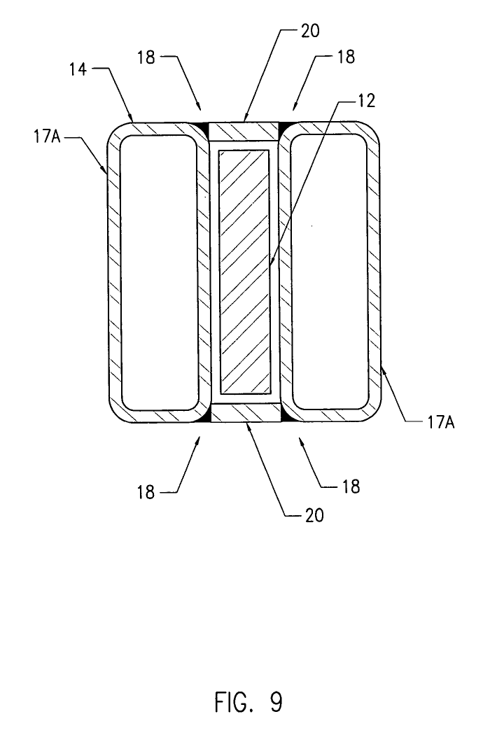

FIG. 9 is a cross-sectional view of another embodiment of the present invention. Here, the shell 14 includes two shell halves comprised of hollow sections 17A. The two hollow sections are connected with connecting plates 20 via shell connection 18. Preferably, the face of hollow sections 17A and the connecting plates 20 do not come into contact with the core 12, but preferably come into close proximity to the core 12.

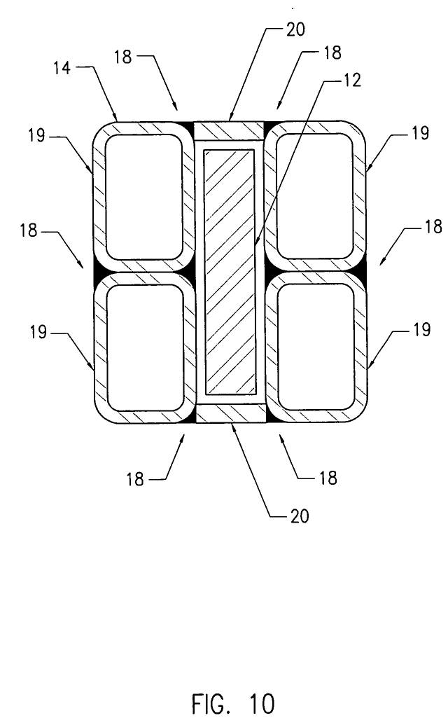

FIG. 10 is a cross-sectional view of another embodiment of the present invention. Here, the shell 14 includes two shell halves, each comprised of plurality of hollow sections 19. The shell halves are connected with connecting plates 20 via shell connection 18. Preferably, the face of hollow sections 19 and the connecting plates 20 do not come into contact with the core 12, but preferably come into close proximity to the core 12.

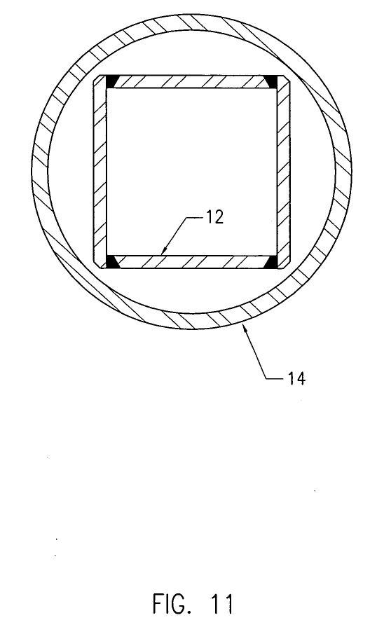

FIG. 11 is a cross-sectional view of another embodiment of the BRSBA 10. Here, a rectangular hollow section core 12 is preferably substantially surrounded by a round tube shell 14.

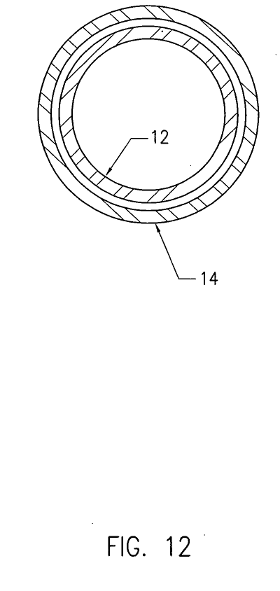

FIG. 12 is a cross-sectional view of another embodiment of the present invention. Here, a round tube core 12 is preferably substantially surrounded by a cylindrical tube shaped shell 14.

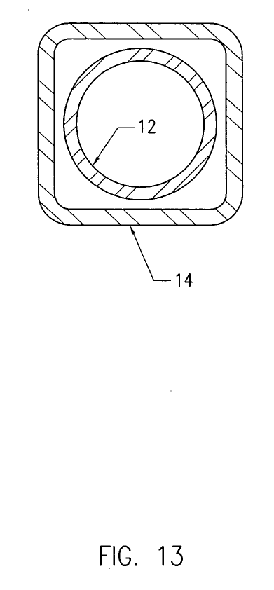

FIG. 13 is a cross-sectional view of another embodiment of the present invention. Here, a round tube core 12 is preferably substantially surrounded by a rectangular tube shaped shell 14.

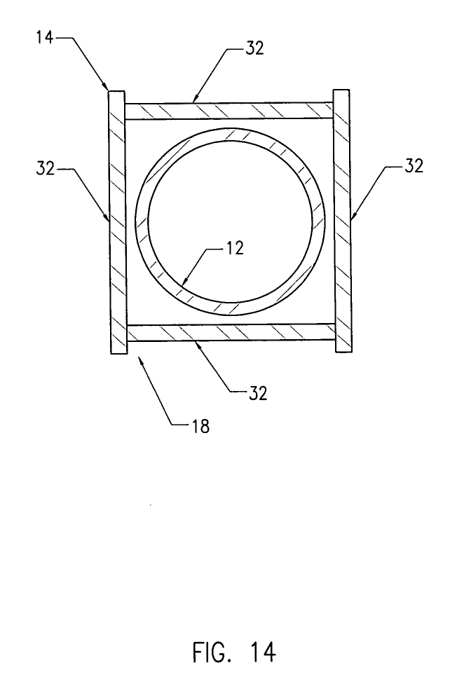

FIG. 14 depicts a cross-sectional view of another embodiment of the present invention. A round tube core 12 is preferably substantially surrounded by a shell 14 made up of plates 32. These plates 32 may be welded or fastened together.

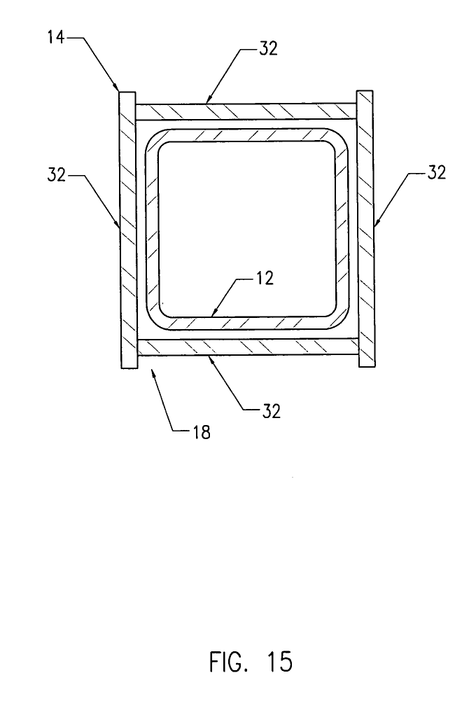

FIG. 15 is a cross-sectional view of another embodiment of the present invention. Here, a rectangular tube core 12 is preferably substantially surrounded by four plates 32. The plates 32 may be welded together or attached by some other fastening method.

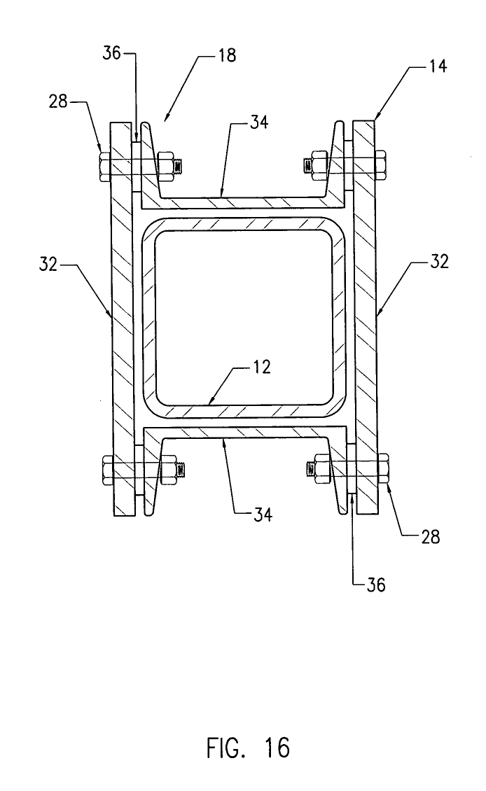

FIG. 16 depicts a cross-sectional view of another embodiment of the present invention. A rectangular tube core 12 is preferably substantially surrounded by a shell 14 made up of plates 32 and channels 34. The plates 32 and channels 34 making up the shell 14 may be welded or attached by some other fastening method. A shim 36 may be included in the shell 14 assembly as needed.

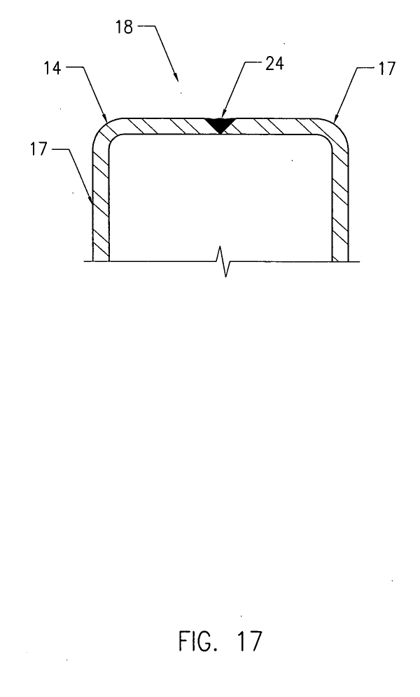

FIG. 17 illustrates a shell connection 18 of the shell 14 in an embodiment of the buckling restrained structural brace assembly (not shown). Two shell halves 17 are connected at their mating surfaces with a weld 24 to form a rigid shell 14 member.

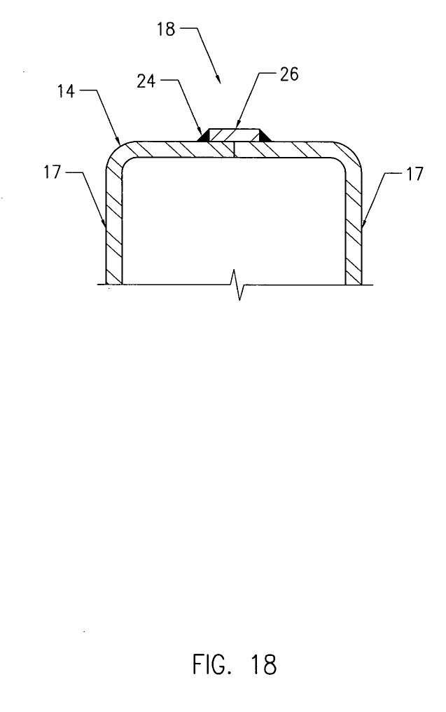

FIG. 18 illustrates an embodiment of a shell connection 18 of the shell 14. Here, the two shell halves 17 are joined using a weld plate 26 with two welds 24 on either lateral edge of the weld plate 26.

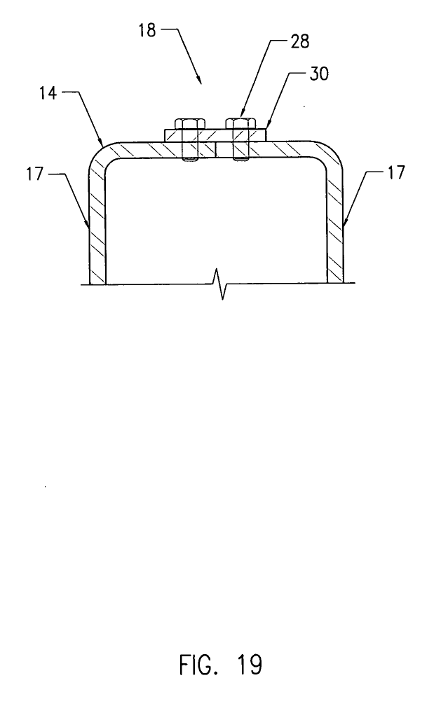

FIG. 19 illustrates a shell connection 18 of the shell 14 in an embodiment of the buckling restrained structural brace assembly (not shown). Here, the two shell halves 17 are connected by way of a bolt plate 30 facilitating the bolted connection 28. The bolted connection 28 passes through the material of the shell halves 17. The through holes (not shown) on the bolt plate 30 and the through holes (not shown) on the two shell halves 17 may be threaded or may be smooth.

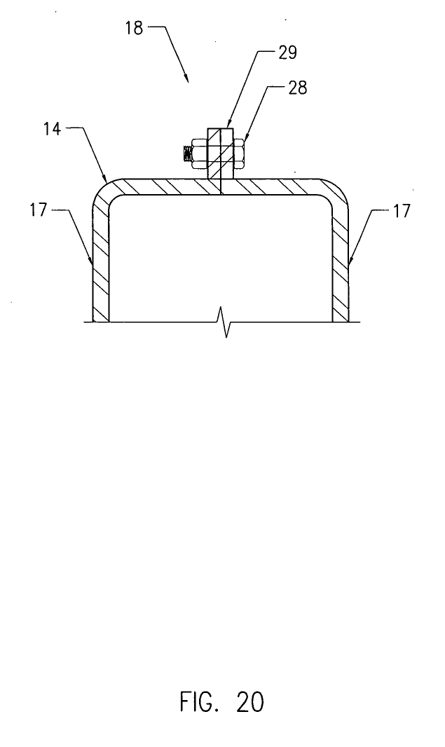

FIG. 20 illustrates a shell connection 18 of the shell 14 in an embodiment of the present invention. Here, the mating surfaces of the two shell halves 17 are comprised of flanges 29 to facilitate a bolted connection 28.

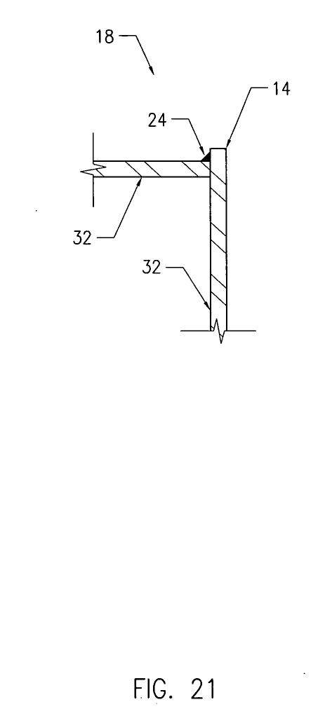

FIG. 21 illustrates an embodiment of a shell connection 18 of the shell 14. Here, two plates 32 are joined together with a weld 24.

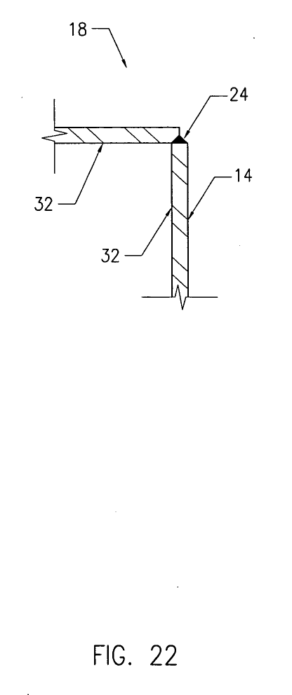

FIG. 22 illustrates another embodiment of a shell connection 18 of the shell 14. Here, two plates 32 are joined together with a weld 24.

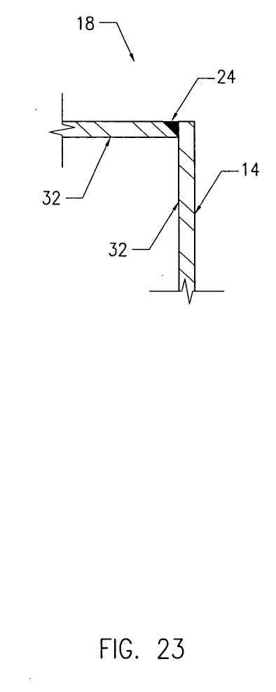

FIG. 23 illustrates an embodiment of a shell connection 18 of the shell 14. Here, two plates 32 are joined together with a weld 24.

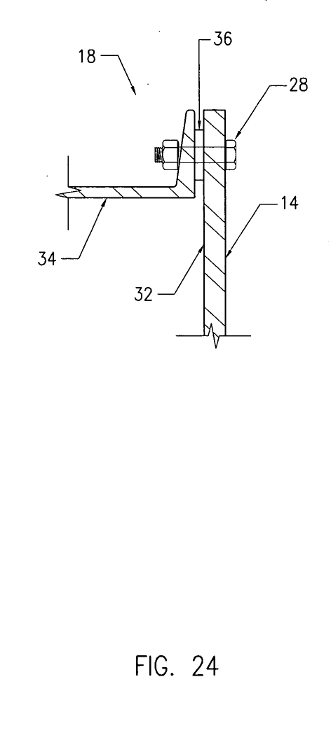

FIG. 24 illustrates another embodiment of a shell connection 18 of the shell 14. Here, a length of channel 34 is connected to a plate 32 by a bolted connection 28. A shim 36 may be used as necessary.

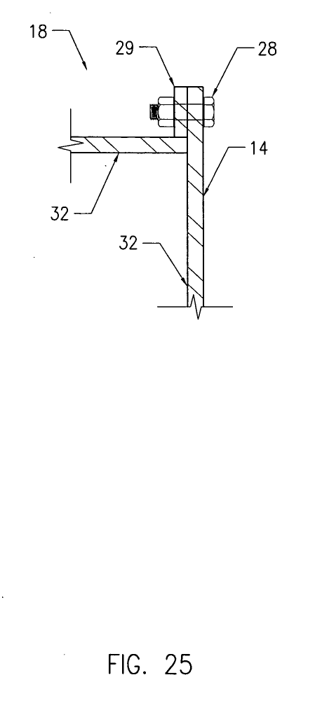

FIG. 25 illustrates an embodiment of a shell connection 18 of the shell 14. A length of channel 34 is connected to a plate 32 by a bolted connection 28. The channel 34 may be fabricated from plates (not shown) to create a flange 29 to facilitate the bolted connection 28.

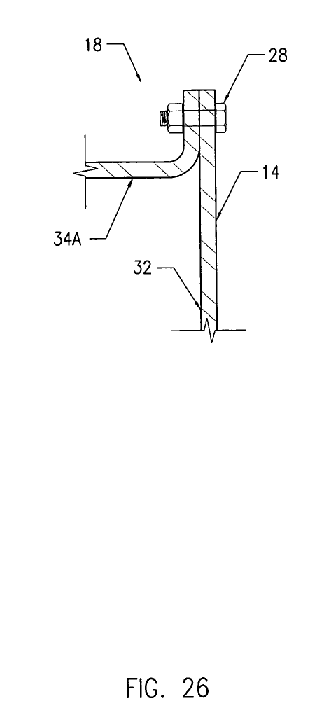

FIG. 26 illustrates another embodiment of a shell connection 18 of the shell 14. A length of channel bent from flat plate 34A is connected to a plate 32 by a bolted connection 28.

By way of example and not by limitation, the following embodiments of the buckling restrained structural brace assembly are contemplated.

An embodiment of the present invention is made up of a brace (core) element enclosed with a hollow section (shell). The brace element may be of rectangular, circular, cruciform, or other double-symmetrical shapes in cross-section. The hollow section may be rolled (seam welded or seamless) or built-up shapes with steel plates. The BRSBA 10 may in an embodiment include other configurations for the shell, including shapes made from plates, channels, and other hollow sections. The configuration of the shell is limited only by functionality. Lateral restraint to the brace element is provided by the surrounding shell, with or without fins attached to the core or the inside surface of the shell. A small gap between the brace and the hollow section is provided to allow the lateral expansion of the brace cross-section due to axial compression in the longitudinal direction of the brace.

Preferably, in an embodiment of the present invention, the cross-sectional dimensions of the core are determined based on the stiffness and strength requirements of the brace for the intended use. The size and configuration of the shell are intended to provide adequate lateral restraint to the brace in order to prevent the brace from buckling. Continuous or segmented fins are installed, as necessary, to bridge the space between the brace and the hollow section. The gap between the fins and the hollow section, or the gap between the fins and the brace, is determined based on material properties of the brace and the anticipated brace compressive deformation.

In an embodiment of the present invention, the load-bearing member and the buckling-restraining member may be composed of steel or other metal. The load-bearing member and the buckling-restraining member may consist of plates, rolled structural sections, hollow structural sections, or any combination of these elements welded or otherwise fastened together to form the buckling restrained structural brace assembly. Preferably, in an embodiment of the present invention, for some configurations of load-bearing member and buckling-restraining member combinations, fins, of similar material, can be attached to the load-bearing member, or to the buckling-restraining member to bridge the space between the load-bearing and buckling-restraining members. A similar gap between the fins, when attached to the load-bearing member, and the buckling-restraining member shall be maintained. Likewise, a similar gap between the fins, when attached to the buckling-restraining member, and the load-bearing member shall be maintained.

Preferably, in an embodiment of the present invention, the buckling restrained structural brace assembly has the advantage of simple fabrication (all components may be made of structural steel that can be fabricated in the same shop), easy assembly and installation, and easy access to the brace core (by removing and re-installing the outer shell) for inspection and replacement.

In an embodiment of the present invention, the buckling-restraining member is preferably separated from the load-bearing member. Preferably the distance between the buckling-restraining member and the load-bearing member would be sufficient to prevent the load-bearing member from interlocking with the buckling-restraining member when the load-bearing member is subjected to continuously increased axial compression and experience maximum expected lateral expansion. In an embodiment of current invention, expansion differs from deflection. Here, expansion is used to describe an increase in any or all dimensions of the cross section of the load-bearing member as axial compression is applied. Deflection, on the other hand, is used to describe the movement of the entire cross section of the load-bearing member in a direction normal to the longitudinal axis, along which axial compression is applied.. Preferably, the distance between the load-bearing member and the buckling-restraining member is only slightly greater than a distance sufficient to prevent the load-bearing member from interlocking with the buckling-restraining member when the load-bearing member is subjected to increased axial compression and experience maximum lateral expansion. In an embodiment of the present invention, this distance is minimized to reduce the total amount of lateral deflection experienced by the load-bearing member.

Preferably, in an embodiment of the present invention, a buckling-restraining member is easily installed around a new brace or load-bearing member, or around an existing brace or load-bearing member in an existing structure. Thus, an embodiment of the present invention could be used to enhance the performance of existing braces.

In an embodiment of the present invention, substances with low friction coefficients may be used on one or both of the opposing surfaces of the gap to limit the stress transfer by friction from the load-bearing member to the buckling-restraining member.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

The invention is claimed as follows:

1. A device for carrying applied load comprising:
a. a load-bearing member;
b. a buckling-restraining member to prevent the load-bearing member from buckling, the buckling-restraining member substantially surrounding the load-bearing member; and
c. a plurality of fins attached to the load-bearing member, the fins being spaced apart from the buckling-restraining member by a distance of less than one inch.

2. The device as recited in claim 1, wherein the load-bearing member is a metal selected from the group consisting of steel and aluminum.

3. The device as recited in claim 1, wherein the buckling-restraining member is a metal selected from the group consisting of steel and aluminum.

4. The device as recited in claim 1, wherein the buckling-restraining member comprises a plurality of openings for inspection of the load-bearing member.

5. The device as recited in claim 1, wherein the load-bearing member comprises a single member or a plurality of members fastened together to carry load primarily in the form of axial tension and compression.

6. The device as recited in claim 1, wherein the buckling-restraining member comprises a plurality of members, fastened together to substantially surround the load-bearing member.

7. A device for carrying applied load comprising:
a. a load-bearing member;
b. a buckling-restraining member to prevent the load-bearing member from buckling, the buckling-restraining member substantially surrounding the load-bearing member; and
c. a plurality of fins attached to the buckling-restraining member, the fins being spaced apart from the load-bearing member by a distance of less than one inch.

8. The device as recited in claim 7, wherein the load-bearing member is a metal selected from the group consisting of steel and aluminum.

9. The device as recited in claim 7, wherein the buckling-restraining member is a metal selected from the group consisting of steel and aluminum.

10. The device as recited in claim 7, wherein the buckling-restraining member comprises a plurality of openings for inspection of the load-bearing member.

11. The device as recited in claim 7, wherein the load-bearing member comprises a single member or a plurality of members fastened together to carry load primarily in the form of axial tension and compression.

12. The device as recited in claim 7, wherein the buckling-restraining member comprises a plurality of members, fastened together to substantially surround the load-bearing member.

13. A device for carrying applied load comprising:
a. a load-bearing member;
b. a buckling-restraining member to prevent the load-bearing member from buckling, the buckling-restraining member substantially surrounding the load-bearing member and being spaced apart from the load-bearing member by a distance of less than one inch.

14. The device as recited in claim 13, wherein the load-bearing member is a metal selected from the group consisting of steel and aluminum.

15. The device as recited in claim 13, wherein the buckling-restraining member is a metal selected from the group consisting of steel and aluminum.

16. The device as recited in claim 13, wherein the buckling-restraining member comprises a plurality of openings for inspection of the load-bearing member.

17. The device as recited in claim 13, wherein the load-bearing member comprises a single member or a plurality of members fastened together to carry load primarily in the form of axial tension and compression.

18. The device as recited in claim 13, wherein the buckling-restraining member comprises a plurality of members, fastened together to substantially surround the load-bearing member.
